(19) United States
(12) Patent Application Publication
(10) Pub. No.: US 2012/0051498 A1
(43) Pub. Date: Mar. 1, 2012

(54) X-RAY IMAGING SYSTEM AND X-RAY IMAGING METHOD

(75) Inventor: Takeshi KOISHI, Kanagawa (JP)

(73) Assignee: FUJIFILM CORPORATION, Tokyo (JP)

(21) Appl. No.: 13/216,157

(22) Filed: Aug. 23, 2011

(30) Foreign Application Priority Data
Aug. 30, 2010 (JP) 2010-192055

(51) Int. Cl. A61B 6/03

(52) U.S. Cl. 378/10

(57) ABSTRACT

An X-ray imaging system is for irradiating a subject with X ray from an X-ray source at a plurality of different angles to acquire X-ray images, and reconstructing an X-ray tomographic image from the X-ray images. The system comprising a region setting unit for setting a region of interest on a pre-shot image or a previously acquired X-ray tomographic image, an imaging control unit for continuously varying an aperture formed by collimator blades according to a position of the X-ray source to image the region of interest and acquiring projection data of the X-ray images in accordance with the position of the X-ray source, and an image reconstructing unit for reconstructing the X-ray tomographic image from the projection data of the X-ray images of the region of interest. The aperture formed by the collimator blades are varied so that all the X-ray images formed on the X-ray detector is rectangular regardless of the position of the X-ray source.

### BACKGROUND OF THE INVENTION

The present invention relates to an x-ray imaging system and an x-ray imaging method for reconstructing an X-ray tomographic image of a subject in a cross section at an arbitrary height thereof using projection data of X-ray images acquired by tomosynthesis imaging.

An X-ray imaging system for tomosynthesis imaging irradiates a subject with X ray at different angles as the X-ray source is moved in one direction and detects the X ray with which the subject has been irradiated using a flat panel X-ray detector (FPD) to achieve acquisition of projection data of X-ray images of the subject taken at different angles in a single imaging operation. Then the process proceeds to image processing using the projection data of the X-ray images to reconstruct an X-ray tomographic image in a cross section of the subject at an arbitrary height thereof.

Now, reconstruction of an X-ray tomographic image will be described.

In tomosynthesis imaging, the X-ray source is moved in one direction to irradiate a subject 30 with X ray from positions S1, S2, and S3 as illustrated in FIG. 5A and obtain X-ray images (projection data) P1, P2, and P3 of the subject 30.

Suppose that there are two objects to be imaged A and B at two positions of the subject 30 different in height. The X ray radiated from the X-ray source at the imaging positions S1, S2, and S3 passes through the subject 30 to enter the FPD. As a result, the two objects to be imaged A and B are projected to different positions in the X-ray images P1, P2, and P3 corresponding to the imaging positions S1, S2, and S3.

In the X-ray image P1, for example, the position S1 of the X-ray source is located to the left of the objects to be imaged A and B with respect to the X-ray source moving direction, so that the objects to be imaged A and B are projected to positions P1A and P1B that are set off to the right of the objects to be imaged A and B. Likewise, in the X-ray image 82, the projections are formed in positions P2A, P2B that are substantially directly beneath the objects A, B; in the X-ray image P3, the Projections are formed in positions P3A, P3B that are set off to the left of the objects A, B.

To reconstruct an X-ray tomographic image of the subject in a cross section at a height of the object to be imaged A, the X-ray image P1 is shifted leftward, and the X-ray image P3 is shifted rightward, for example, so that the projection positions P1A, P2A, and P3A coincide as illustrated in FIG. 5B (shift addition method). Thus, an X-ray tomographic image is reconstructed wherein the cross section located at the height of the object to be imaged A is enhanced. An X-ray tomographic image in a cross section located at an arbitrary height containing a cross section at a height of the object to be imaged B may likewise be reconstructed.

However, tomosynthesis imaging, whereby a plurality of X-ray images are acquired in a single imaging operation, had a problem of an increased dosage for a subject. To solve such problem, there has been made a proposition that a collimator be used to irradiate only a region of interest with X ray in order to avoid unnecessary exposure of the peripheral region to X ray.

JP 2008-012319 A, for example, describes a tomosynthesis system for effecting an improved reduction of artifacts and comprising an X-ray source for projecting an X-ray beam from a plurality of positions through an object to be imaged and a collimator disposed between the X-ray source and the object to be imaged, so that regions of interest in a plurality of projection images are defined after acquisition of image data based on an at least previously defined region, and the regions of interest in the respective projection images are back-projected to reconstruct at least one 3D image.

### SUMMARY OF THE INVENTION

However, according to the method described in JP 2008-012319 A, the field of view in the projection image is determined by the collimator, and the region of interest is determined by the operator, whereas the region of interest is defined from the projection image based on the field of view of the collimator, and the volumetric image of the scanned volume is formed using reconstruction algorithm from the projection image contained within the range of the region of interest. However, the literature does not describe continuously varying the collimator blades according to the position of the X-ray source to ensure that the X-ray images on the FPD are rectangular regardless of the position of the X-ray source.

An object of the present invention is to provide an X-ray imaging system and an X-ray imaging method for reconstructing an X-ray tomographic image in a cross section at an arbitrary height of the subject, the X-ray imaging system and the X-ray imaging method enabling reduction in exposure dosage, shortening imaging time, and reduction of blurring of an image due to a movement of a patient by irradiating only a region of interest with X ray.

In order to attain the object described above, the present invention provides an X-ray imaging system for irradiating a subject with X ray from an X-ray source at a plurality of different angles, detecting the X ray transmitted through the subject with an X ray detector to acquire X-ray images, and reconstructing an X-ray tomographic image, which is a tomosynthesis image, from the X-ray images, the system comprising:

a region setting unit for setting a region of interest on a pre-shot image or a previously acquired X-ray tomographic image,

an imaging control unit for continuously varying an aperture formed by collimator blades according to a position of the X-ray source to image the region of interest and acquiring projection data of the X-ray images in accordance with the position of the X-ray source, and

an image reconstructing unit for reconstructing the X-ray tomographic image from the projection data of the X-ray images of the region of interest,

wherein the aperture formed by the collimator blades are varied so that all the X-ray images formed on the X-ray detector is rectangular regardless of the position of the X-ray source.

Also, the present invention provides an X-ray imaging method of irradiating a subject with X ray from an X-ray source at a plurality of different angles, detecting the X ray transmitted through the subject with an X ray detector to acquire X-ray images, and reconstructing an X-ray tomographic image, which is a tomosynthesis image, from the X-ray images, the method comprising:

a region setting step of setting a region of interest on a pre-shot image or a previously acquired X-ray tomographic image,

an imaging control step of continuously varying an aperture formed by collimator blades according to a position of the X-ray source to image the region of interest and acquiring projection data of the X-ray images in accordance with the position of the X-ray source so that all the X-ray images formed on the X-ray detector is rectangular regardless of the position of the X-ray source, and

an image reconstructing step of reconstructing the X-ray tomographic image from the projection data of the X-ray images of the region of interest.

The present invention enables acquisition of X-ray images for reconstructing an X-ray tomographic image with a small exposure dosage, and improvement in imaging cycle enables shortening of the imaging time. A shortened imaging time in turn reduces blurring of images due to a movement of a patient, and reconstruction of an X-ray tomographic image is achieved from clearer X-ray images.

### BRIEF DESCRIPTION OF THE DRAWINGS

FIG. 1 is a block diagram representing a configuration of an embodiment of an X-ray imaging system according to the invention.

FIG. 2 is a view for explaining a relationship between an original region of interest and a region of interest.

FIG. 3 is a view for explaining a relationship between the position of an X-ray source and an aperture formed by collimator blades.

FIG. 4 is a flowchart illustrating an example of a processing flow in an X-ray imaging system of the invention.

FIGS. 5A and 5B are conceptual views illustrating reconstruction of an X-ray tomographic image in tomosynthesis imaging.

### DETAILED DESCRIPTION OF THE INVENTION

The X-ray imaging system of the invention for implement an X-ray imaging method of the invention will be described in detail based upon the preferred embodiments illustrated in the attached drawings.

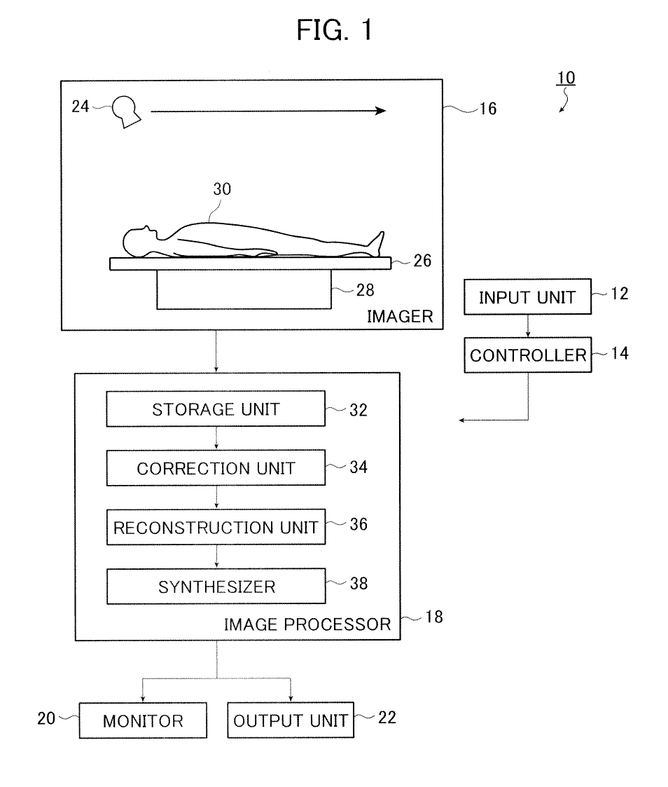

FIG. 1 is a block diagram representing a configuration of an embodiment of an X-ray imaging system according to the invention.

An X-ray imaging system 10 illustrated in FIG. 1 acquires images of a subject 30 such as a human body by tomosynthesis imaging (X-ray imaging) and reconstructs an X-ray tomographic image in a cross section located at an arbitrary height of the subject 30. The X-ray imaging system 10 comprises an input unit 12, a controller 14, an imager 16, an image processor 18, a monitor 20, and an output unit 22.

The input unit 12 is provided to enter various instructions including but not limited to an instruction to start imaging and may be configured by a mouse, a keyboard, etc. The input unit 12 produces an instruction signal, which is received by the controller 14.

The controller 14 produces control signals according to an instruction signal entered from the input unit 12 to control the operations of the X-ray imaging system 10 including imaging operations and setting of a region of interest by the imager 16, image processing by the image processor 18, screen display by the monitor 20, and output processing by the output unit 22. The control signal, not shown, outputted from the controller 14 is entered in the imager 16, the image processor 18, the monitor 20, and the output unit 22.

The region of interest is entered and set by a radiographer using a mouse and the like of the input unit 12 on a pre-shot image displayed on the monitor 20 or an X-ray tomographic image previously acquired, while the thickness of the X-ray tomographic in is set and outputted as region-of-interest information, which forms a part of the control signal. Thus, the input unit 12, the controller 14, and the monitor 20 constitute a region-of-interest setting unit.

The imager 16 acquires images of the subject 30 by tomosynthesis imaging according to a control signal supplied from the controller 14; it comprises an X-ray source 24, a carrier (not shown) for moving the X-ray source 24, an imaging table 26, and a flat panel type X-ray detector (FPD) 28.

The X-ray source 24 comprises an X-ray tube and a collimator and is disposed above and a given distance away from the subject 30 that is positioned on the top surface of the imaging table 26. The X ray radiated from the X-ray tube has its irradiation region limited to the region of interest by the collimator.

The FPD 28 is disposed on the bottom side of the imaging table 26 with its X-ray receiving surface facing upwards. The FPD 28 detects incoming X-ray passing through the subject 30 and effects photoelectric conversion to produce digital image data (projection data) corresponding to an acquired X-ray image of the subject 30. Accordingly, the FPD 28 used in the invention may be a direct type whereby radiation is directly converted into an electric charge, an indirect type whereby radiation is temporarily converted into light, which is then converted into an electric signal, or any of various other types. The FPD 28 may be configured so that it is movable in the same direction as the X-ray source 24.

In tomosynthesis imaging, the carrier controls the movement of the X-ray source 24 to move in one direction and change the X-ray radiation angle toward the subject 30 so as to irradiate the subject 3C with X ray at different imaging angles (at given time intervals). The X-ray radiated by the X-ray source 24 passes through the subject 30 to enter the light receiving surface of the FDP 28, and the FPD 28 converts the X ray into electricity to obtain projection data corresponding to the acquired X-ray images of the subject 30.

In tomosynthesis imaging are acquired a plurality of X-ray images, say 20 to 80 images, of the subject 30, each taken at different imaging angles, in a single imaging operation, whereupon the FPD 28 sequentially outputs projection data corresponding to the acquired X-ray images.

Thus, the controller 14 and the imager 16 constitute an imaging unit.

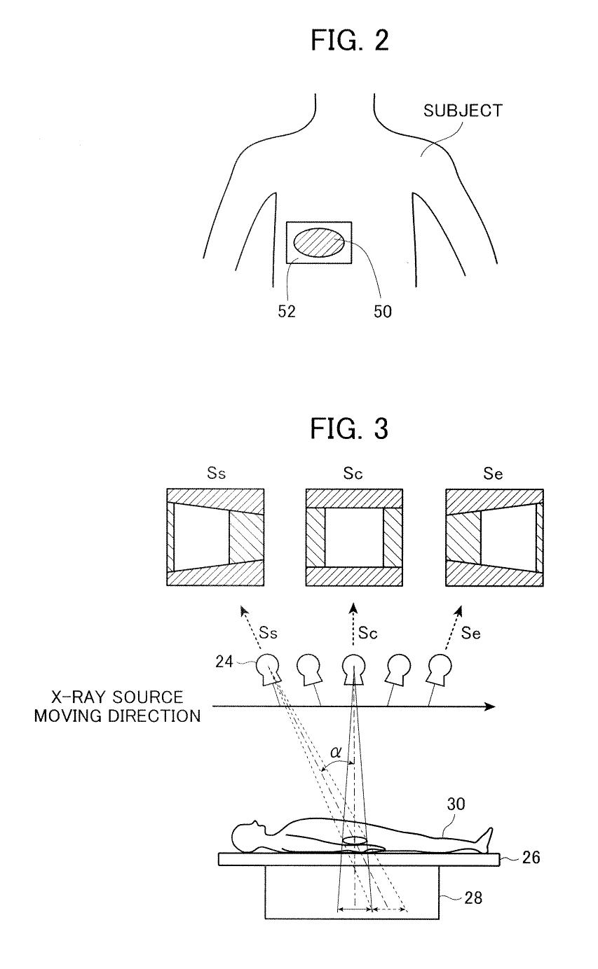

Now, the region of interest will be described referring to FIG. 2. The figure illustrates an original region of interest 50 of the subject and a region of interest 52 for the sake of explanation, which contains the original region of interest 50 and represents a smallest possible range that can be set by the collimator, that is, a restricted imaging range. The region of interest 52 is preferably set by a rectangle so as to be easy for the radiographer to enter data using a mouse or the like of the input unit 12 but may be a circle or an ellipse. The thickness of the X-ray tomographic image is also set.

FIG. 3 illustrates a relationship between the position of the X-ray source 24 and the aperture formed by the collimator blades.

The X-ray source 24 moves from left to right as indicated by an arrow in the figure following a straight line trajectory at a constant speed to acquire a plurality of X-ray images. The FPD 28 is fixed in position.

In this case, Ss indicates a first imaging position, Sc indicates an imaging position precisely above the region of interest (i.e., center of the movement range of the X-ray source 24), and Se indicates a last imaging position. At the imaging position Ss, since the X ray irradiates the region of interest at an angle, the aperture formed by the collimator blades have the shape of a trapezoid having a smaller width on the side closer to the region of interest, so that the X-ray image formed on the FPD 28 is rectangular, of which a center line of a height direction is parallel to the moving direction of the X-ray source 24. In other words, the collimator blades substantially parallel to the moving direction are so controlled as to open wider on the side thereof farther from the region of interest and reduce the aperture on the side closer to the region of interest.

At the imaging position Sc, since X ray irradiates the region of interest from precisely above, the collimator blades form a rectangular aperture similar to the X-ray image on the FPD 28, so that the X-ray image formed on the FPD 28 is rectangular.

At the imaging position Se, since X ray irradiates the region of interest at an angle as at the imaging position Ss, the aperture formed by the collimator blades have the shape of a trapezoid having a smaller width on the side closer to the region of interest, so that the X-ray image formed on the FPD 28 is rectangular, of which the longitudinal center line is parallel to the moving direction of the X-ray source 24. In other words, the apertures formed by the collimator blades at the imaging positions Ss and Se are axisymmetrical with respect to the line perpendicular to the moving direction of the X-ray source and passing through the imaging position Sc (i.e., the line passing through the midpoint of the line connecting the imaging starting position and the imaging ending position). Further, the aperture formed by the collimator blades is axisymmetrical with respect to the straight line trajectory of the X-ray source 24.

The imaging positions from Ss to Se may be obtained from an angle α formed by the straight line connecting the X-ray source 24 at the imaging position Ss and the center of the region of interest on the one hand and the straight line connecting the X-ray source 24 at the imaging position Se and the center of the region of interest according to the thickness of the X-ray tomographic image and the number of X-ray images to be acquired both contained in the region-of-interest information. Thus, the imaging position may be obtained from the angle α and the number of X-ray images to be acquired.

Further, since the relative positions of the X-ray source 24 and the region of interest vary among the imaging positions, the collimator blades are controlled according to the SIT, the angle α, the number of X-ray images to be acquired, and the moving speed of the X-ray source, so that the shape of the aperture formed by the collimator blades at the respective imaging positions continuously varies from a trapezoid, a rectangle, and a trapezoid turned upside down (reversed trapezoid) as seen from the imaging position Ss toward the moving direction of the X-ray source 24.

Then, the image processor 18 receives the projection data produced from the X-ray images acquired by the imager 16 according to the control signal supplied from the controller 14 and performs image processing (including correction and image synthesis) using the projection data produced from the X-ray images to reconstruct an X-ray tomographic image of the subject 30 in a cross section thereof at an arbitrary height. Thus, the image processor 18 is an image reconstructing unit. The image processor 18 comprises a storage unit 32, a correction unit 34, and a reconstruction unit 36, and a synthesizer 38.

The storage unit 32 stores the projection data of the X-ray images acquired by the imager

The correction unit 34 performs various kinds of corrections such as offset correction, residual image correction, gain correction, faulty pixel correction, step correction, longitudinal inconsistent density correction, and lateral inconsistent density correction on the projection data of the X-ray images stored in the storage unit 32. The offset correction, residual image correction, gain correction, faulty pixel correction, step correction, longitudinal inconsistent density correction, and lateral inconsistent density correction are known corrections and may be implemented each using any of various methods including known methods.

The reconstruction unit 36 uses the projection data of the X-ray images corrected by the correction unit 34 to perform image synthesis and reconstruct an X-ray tomographic image of the subject 30 in a cross section thereof at an arbitrary height.

The synthesizer 38 combines the X-ray tomographic image of which only the region of interest has been reconstructed and a pre-shot image of the subject or an X-ray tomographic image previously acquired. For example, the X-ray tomographic image may be synthesized by combining a pre-shot image representing the whole abdomen of the subject with a region of interest narrowed down to a site corresponding to a specific organ. Alternatively, in a case where the whole hand of the subject including a fractured wrist is imaged at a first imaging, and later on another day, only the wrist, which is the region of interest, is imaged in a follow-up examination, the portion of the image representing the wrist, acquired as the region of interest at the second imaging, may be combined, with the X-ray tomographic image representing the whole hand of the subject acquired at the first (previous) imaging to produce a single X-ray tomographic image.

The image processor 18 may be configured by hardware (a device) or a program for causing a computer to execute a part the X-ray image processing method of the invention.

The monitor 20 displays a X-ray tomographic image reconstructed by the image processor 18 according to the control signal supplied from the controller 14 and may be exemplified by a flat panel display such as a liquid crystal display.

The output unit 22 outputs the X-ray tomographic image reconstructed by the image processor 18 according to the control signal supplied from the controller 14 and may be exemplified, for example, by a thermal printer for printing out the X-ray tomographic image and a storage device capable of storing digital image data of the X-ray tomographic image in any of various recording media.

Next, the operation of the X-ray imaging system 10 of the invention for implementing the X-ray imaging method of the invention will be described.

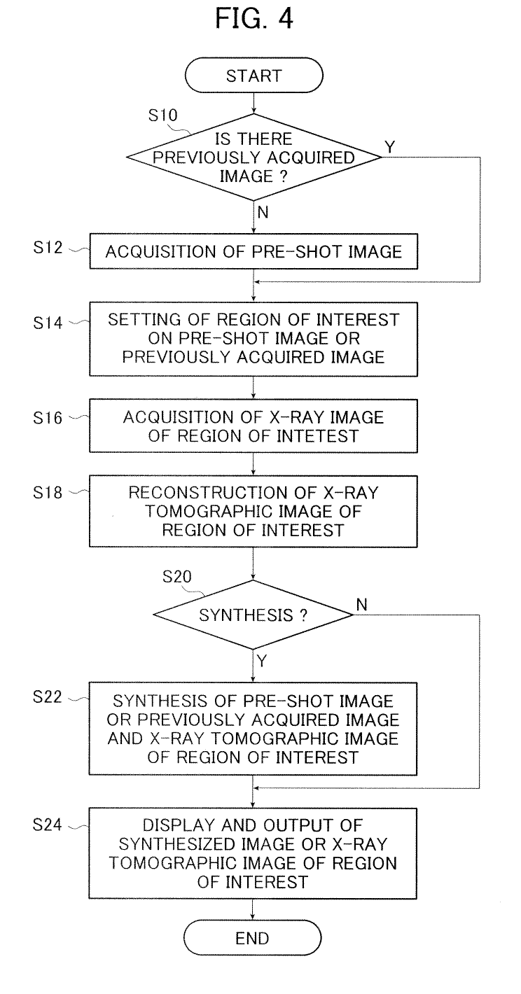

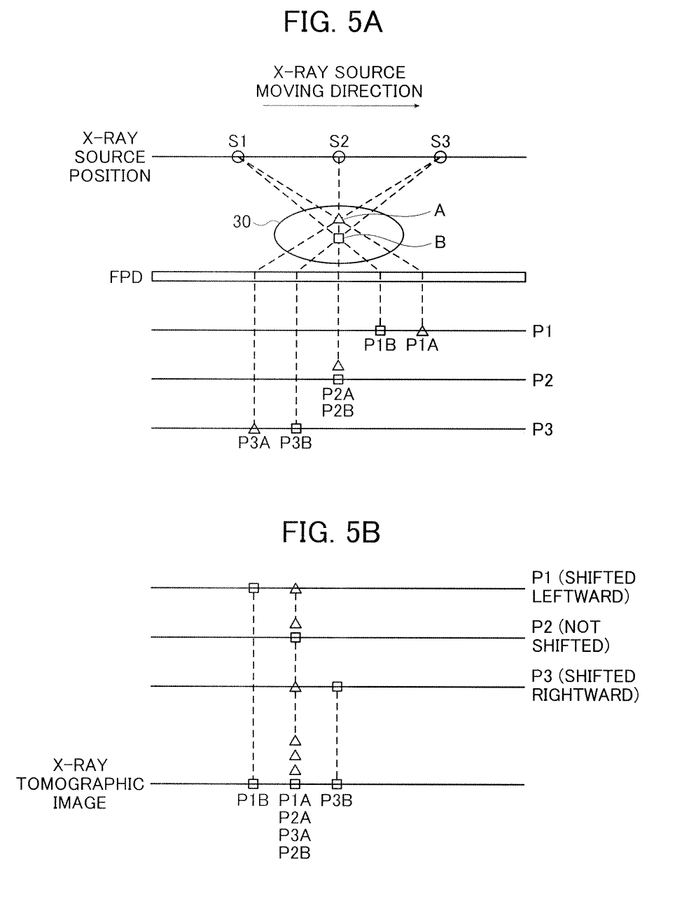

FIG. 4 is a flowchart illustrating an example of a processing flow in the X-ray imaging method of the invention.

Upon entry of an instruction signal for initiating imaging from the input unit 12, the controller 14 judges whether there is an X-ray tomographic image previously acquired representing the same site of the subject based on information such as imaging sites (step S10). The controller 14 searches the X-ray tomographic image data on images acquired in the past and stored in a storage unit constituting a part of the output unit 22 for X-ray tomographic image data representing the same imaging site of the subject, and reads previously acquired X-ray tomographic image data, if any, representing the same imaging site of the subject (Y in step S10).

When the controller 14 finds no X-ray tomographic image data representing the same imaging site of the subject after searching for X-ray tomographic image data representing the same imaging site of the subject, the controller 14 (N in step S10) outputs a control signal to the imager 16 for causing the imager 16 to acquire a pre-shot image covering a range enabling recognition of the position of the region of interest of the subject (e.g., an image representing the whole abdomen), whereupon a pre-shot image is acquired (step S12).

The pre-shot image or the previously acquired X-ray tomographic image is displayed on the monitor 20. Now, the radiographer sets the region of interest on the pre-shot image or the previously acquired X-ray tomographic image, and the region-of-interest information is entered from the input unit 12 (step S14).

The controller 14 produces information on the imaging positions of the X-ray source 24 corresponding to the respective X-ray images and control information related to the collimator blades in accordance with the imaging positions based on the region-of-interest information, whereas the imager 16 varies the radiation angle of the X-ray source 24 so as to direct the X-ray source 24 toward the subject 30 while moving the X-ray source 24 in one direction under the control by the moving mechanism in response to the imaging initializing instruction so as to irradiate the subject 30 with X ray at a different radiation angle and successively acquire X-ray images at different radiation angles starting at the imaging starting position in a single imaging operation (step S16). Each time an X-ray image of the subject 30 is acquired, the FPD 28 produces projection data corresponding to the X-ray image acquired.

At this time, the shape of the aperture formed by the collimator blades changes from a trapezoid to a rectangle, and from a rectangle to a reversed trapezoid, so that the X-ray image formed on the FPD 28 is always rectangular. Alternatively, only the length of the sides of the X-ray images on the FPD 28 parallel to the moving direction of the X-ray source 24 may be allowed to change, and the sides of the X-ray images perpendicular to the moving direction of the X-ray source 24 may be kept from changing, or still alternatively, the collimator blades may be controlled so that all the sides of the X-ray image on the FPD 28 may be kept from changing (so that the size and the aspect ratio of all the X-ray images are substantially the same).

In the image processor 18, the storage unit 32 stores the projection data of the X-ray images of the region of interest acquired by the imager 16, and the correction unit 34 performs various corrections such as offset correction and residual image correction.

The reconstruction unit 36 uses the projection data of the X-ray images of the region of interest corrected by the correction unit 34 to reconstruct an X-ray tomographic image of the region of interest of the subject 30 in a cross section thereof at an arbitrary height (step S18).

Subsequently, when the X-ray tomographic image of the region of interest is combined with the pre-shot image or the previously acquired image (Y in step S20), the synthesizer 38 combines the X-ray tomographic image of the region of interest acquired this time with the region of interest of the pre-shot image or the previously acquired image and outputs the synthesized image as an image for output (step S22). The synthesis by the synthesizer 38 may be a mere replacement of the region of interest of the pre-shot image or the previously acquired image with the X-ray tomographic image of the region of interest acquired this time, or a treatment may be performed to obscure the boundary.

When the X-ray tomographic image of the region of interest is not to be combined with the pre-shot image or the previously acquired image (N in step S20), the synthesizer 38 outputs the X-ray tomographic image of the region of interest as it is as image for output.

The image for output (X-ray tomographic image) outputted from the synthesizer 38 is displayed on the monitor 20. Under the control by the controller 14, the monitor 20 displays the information on the display state of the X-ray tomographic image (information indicating whether synthesis with the pre-shot image or the previously acquired image has been effected).

The image for output (X-ray tomographic image) outputted from the synthesizer 38 is supplied to the output unit 22, which, for example, prints out the X-ray tomographic image and allows digital image data of the X-ray tomographic image to be stored in a recording medium.

Thus, imaging of only the region of interest enables acquisition of X-ray images for reconstructing an X-ray tomographic image with a small exposure dosage, and improvement in imaging cycle enables shortening of the imaging time. A shortened imaging time in turn reduces blurring of images due to a movement of a patient, and reconstruction of an X-ray tomographic image is achieved from clearer X-ray images.

Further, display of the pre-shot image or the previously acquired image as combined with the X-ray tomographic image of the region of interest helps reduce a feeling of incongruity when observing the image. Further, an X-ray tomographic image that is more precise and permits easy recognition of the relative position of the imaging site may be provided.

While the X-ray imaging system and the X-ray imaging method the present invention have been described in detail, the above embodiments are only illustrative and various changes and modifications may be made without departing from the true spirit and scope of the invention.

What is claimed is:

1. An X-ray imaging system for irradiating a subject with X ray from an X-ray source at a plurality of different angles, detecting the X ray transmitted through the subject with an X ray detector to acquire X-ray images, and reconstructing an X-ray tomographic image, which is a tomosynthesis image, from the X-ray images, the system comprising:
a region setting unit for setting a region of interest on a pre-shot image or a previously acquired X-ray tomographic image,
an imaging control unit for continuously varying an aperture formed by collimator blades according to a position of the X-ray source to image the region of interest and acquiring projection data of the X-ray images in accordance with the position of the X-ray source, and
an image reconstructing unit for reconstructing the X-ray tomographic image from the projection data of the X-ray images of the region of interest,
wherein the aperture formed by the collimator blades are varied so that all the X-ray images formed on the X-ray detector is rectangular regardless of the position of the X-ray source.

2. The X-ray imaging system according to claim 1, wherein the aperture formed by the collimator blades has a shape of a trapezoid and a reversed trapezoid when the X-ray source is located at an imaging starting position and an imaging ending point, respectively.

3. The X-ray imaging system according to claim 1, wherein the X-ray images formed on the X ray detector according to the respective positions of the X-ray source change only in length of sides that are parallel to a moving direction of the X-ray source and does not change in length of sides that are perpendicular to the moving direction of the X-ray source.

4. The X-ray imaging system according to claim 1, wherein the X-ray tomographic image is embedded and displayed in the region-of-interest of the pre-shot image or the previously acquired X-ray tomographic image.

5. The X-ray imaging system according to claim 1, wherein the X-ray source moves on a straight line trajectory from an imaging starting position to an imaging ending position.

6. The X-ray imaging system according to claim 1, wherein the X-ray source moves at a constant speed from the imaging starting position to the imaging ending position.

7. The X-ray imaging system according to claim 1, wherein the region of interest is designated by a rectangle.

8. The X-ray imaging system according to claim 7, wherein a thickness of the X-ray tomographic image is further specified, so that the region of interest is designated by a rectangular parallelepiped.

9. The X-ray imaging system according to claim 1, wherein the apertures formed by the collimator blades at an imaging starting position and an imaging ending position are axisymmetrical with respect to a line perpendicular to the moving direction of the X-ray source passing through a midpoint of a line connecting the imaging starting position and the imaging ending position.

10. An X-ray imaging method of irradiating a subject with X ray from an X-ray source at a plurality of different angles, detecting the X ray transmitted through the subject with an X ray detector to acquire X-ray images, and reconstructing an X-ray tomographic image, which is a tomosynthesis image, from the X-ray images, the method comprising:
a region setting step of setting a region of interest on a pre-shot image or a previously acquired X-ray tomographic image,
an imaging control step of continuously varying an aperture formed by collimator blades according to a position of the X-ray source to image the region of interest and acquiring projection data of the X-ray images in accordance with the position of the X-ray source so that all the X-ray images formed on the X-ray detector is rectangular regardless of the position of the X-ray source, and
an image reconstructing step of reconstructing the X-ray tomographic image from the projection data of the X-ray images of the region of interest.
